(19) United States
(12) Plant Patent Application Publication
(10) Pub. No.: US 2012/0260385 P1
(43) Pub. Date: Oct. 11, 2012

(54) Sutera plant named 'DANCOP37'

(50) ;

(76) Inventor: Gavriel Danziger, Moshav Mishmar Hashiva (IL)

(21) Appl. No.: 13/066,204

(22) Filed: Apr. 7, 2011

(51) Int. Cl. A01H 5/00

(52) U.S. Cl. PLT/485

(57) ABSTRACT

A new and distinct Sutera cultivar named ‘DANCOP37’ is disclosed, characterized by extra large blue flowers, early and abundant flowering. The new variety has a semi-trailing, compact growth habit and moderate vigor. The new variety is a Sutera, normally produced as an outdoor garden or container plant.

### LATIN NAME OF THE GENUS AND SPECIES

Sutera cordata

### VARIETY DENOMINATION

‘DANCOP37’

### BACKGROUND OF THE INVENTION

The new Sutera cultivar is a product of a planned breeding program conducted by the inventor, Gavriel Danziger, in Moshav Mishmar Hashiva, Israel. The objective of the breeding program was to produce new Sutera varieties for ornamental commercial applications. The cross resulting in this new variety was made during Jun. 2007.

The seed parent is the unpatented, proprietary seedling variety referred to as Sutera cordata ‘Cv-07-577’ The pollen parent is unknown, as the crossing resulting in ‘DANCOP37’ was an open pollination, with unidentifiable pollen parents. The new variety was discovered in April 2008 by the inventor in a group of seedlings resulting from the 2007 crossing, in a research greenhouse in Moshav Mishmar Hashiva, Israel.

Asexual reproduction of the new cultivar ‘DANCOP37’ by vegetative cuttings was first performed at a research greenhouse in Moshav Mishmar Hashiva, Israel in May 2008 and has shown that the unique features of this cultivar are stable and reproduced true to type in successive generations.

### SUMMARY OF THE INVENTION

The cultivar ‘DANCOP37’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘DANCOP37’ These characteristics in combination distinguish ‘DANCOP37’ as a new and distinct Sutera cultivar:

1. Semi-trailing, compact growth habit

2. Extra large lue flowers

3. Very free branching plants

4. Early, abundant flowering

5. Moderately vigorous growth

Plants of the new cultivar ‘DANCOP37’ are similar to plants of the seed parent, Sutera cordata ‘PCv07-577’ in most horticultural characteristics, however, plants of the new cultivar ‘DANCOP37’ have a somewhat less trailing, more compact plant habit, larger flowers of a different color, and more branches per plant. Additionally, the new cultivar begins flowering earlier, produces more flowers, and slightly less vigorous plants than the seed parent.

### COMMERCIAL COMPARISON

Plants of the new cultivar ‘DANCOP37’ are comparable to the variety Sutera ‘DANCOP24’ U.S. Plant Pat. No. 19343. The two Sutera varieties are similar in most horticultural characteristics however, the new variety ‘DANCOP37’ differs in having different color flowers, and an overall more compact plant habit. Additionally, the new variety has larger leaves than ‘DANCOP24’.

Plants of the new cultivar ‘DANCOP37’ can also be compared to the commercial variety Sutera ‘DANGUL14’ U.S. Plant Pat. No. 18194. These varieties are similar in most horticultural characteristics, however ‘DANCOP37’ has different color flowers and slightly more branching than ‘DANGUL14’. Additionally, plants of ‘DANCOP37’ grow somewhat less vigorously than DANGUL14’.

### BRIEF DESCRIPTION OF THE PHOTOGRAPHS

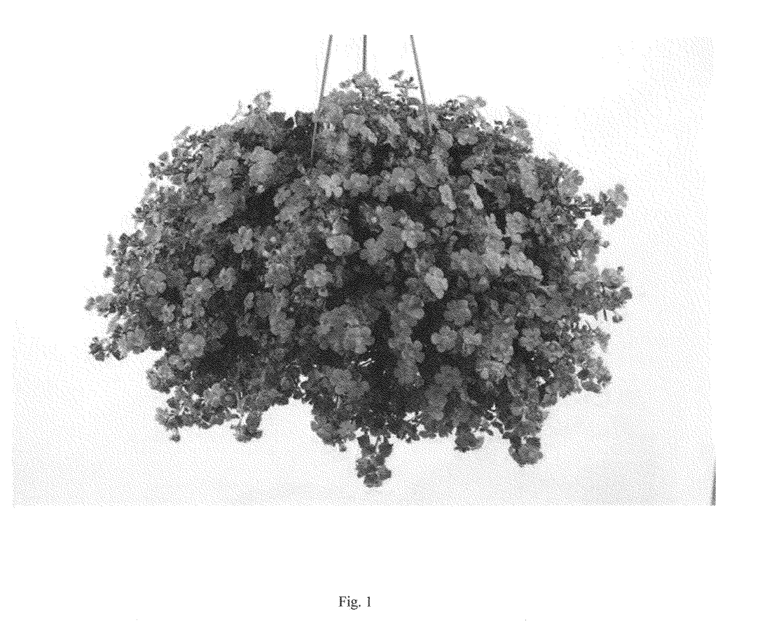

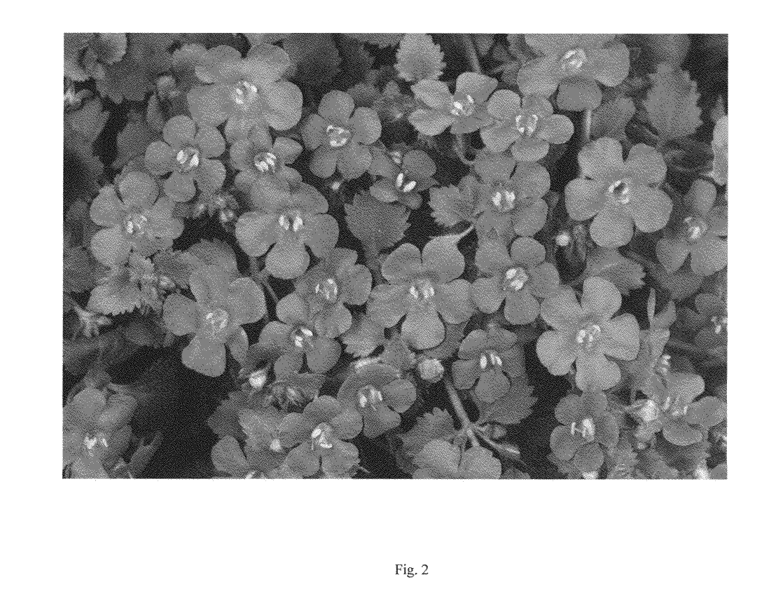

The accompanying photograph in FIG. 1 illustrates in full color a typical plant of ‘DANCOP37’ grown in a greenhouse, in a 13 cm basket. Age of the plant photographed is approximately 3 months from a rooted cutting. FIG. 2 illustrates in full color a close up of a typical bloom of ‘DANCOP37’. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2001 except where general terms of ordinary dictionary significance are used. The following observations and measurements describe ‘DANCOP37’ plants grown in a greenhouse during Winter and Spring in Moshav Mishmar Hashiva, Israel. The growing temperature ranged from 15° C. to 26° C. during the day and from 13° C. to 16° C. during the night. General light conditions are bright, normal sunlight. Measurements and numerical values represent averages of typical plant types.
Botanical classification: Sutera cordata ‘DANCOP37 ’
Propagation:
   Time to initiate roots.—About to 10 days at approximately 20° C.
   Root description.—Fine, fibrous.
Plant:
   Growth habit.—Semi-trailing, compact.
   Pot size of plant described.—13 cm.
   Height.—Approximately 10 cm.
   Plant spread.—approximately 40 cm.
   Growth rate.—Moderate vigor.
   Branching characteristics.—Very free branching.
   Length of primary lateral branches.—Approximately 30 cm.
   Diameter of lateral branches.—Approximately 0.2 cm.
   Quantity of primary lateral branches.—Approximately 10.
   Characteristics of primary lateral branches.—Form: Cylindrical Diameter: Approximately 0.2 cm. Color: Near RHS Green 138 B Texture: Velvety Strength: Strong.
   Internode length.—Approximately 2 cm.
Foliage:
   Leaf.—Arrangement: Opposite, petiolate Quantity: Approximately 50 per branch. Average Length: 3.5 cm. Average Width: 2.5 cm. Shape of blade: Ovate Apex: Acute Base: Acuminate Margin: Serrate Texture of top surface: velvety Pubescence: Slightly pubescent Aspect: Flat Color: Young foliage upper side: RHS Green 137 C Young foliage under side: RHS Green 137 D Mature foliage upper side: RHS Green 137 A Mature foliage under side: RHS Green 137 B Venation: Type: Pinnate. Venation color upper side: RHS Green 137 A Venation color under side: RHS Green 137 B Petiole: Length: Approximately 0.5 cm. Diameter: Approximately 0.1 cm. Color: RHS Green 137 A Texture: Slightly velvety.
Flower:
   Natural flowering season.—All year round.
   Days to flowering from rooted cutting.—about 45.
   Inflorescence and flower type and habit.—Axillary, solitary salverform single flowers.
   Rate of flower opening.—3 to 5 days from bud to fully opened flower.
   Flower longevity on plant.—about 7 days.
   Persistent or self-cleaning.—Persistent.
   Bud.—Shape: Spatulate Length: about 1.2 cm Diameter: about 0.6 cm Color: RHS Violet-Blue 93 C.
   Flower size.—Diameter: about 2.9 cm. Length: about 1.5 cm. Corolla tube diameter: about 0.5 cm.
   Corolla/petals.—Arrangement: Symmetric Lobe Length: about 1.0 cm. Lobe Width: about 1.2 cm. Quantity: 5 Texture: Velvety Lobe Apex: Rounded Lobe shape: Rounded Margin: Entire.
   Color.—When opening: Upper surface: RHS Violet N 88 A Lower surface: RHS Violet N 88 C Fully opened: Upper surface: RHS. Violet N 88 B Lower surface: RHS Violet N 88 C Visible Corolla tube color: RHS Yellow-Orange 21 A.
   Calyx/sepals.—Quantity per flower: 5 Shape: Linear Length: About 0.6 cm Width: About 0.1 cm Apex: Acute Base: Fused. Margin: Entire Texture: Slightly velvety Color: Upper Surface: RHS Green 137 B Lower Surface: RHS Green 137 B.
   Peduncle.—None.
   Pedicel.—Length: Approximately 1 cm. Diameter: Approximately 0.1 cm. Color: Near RHS Green 138 B Orientation: Slanted upward and outward Pubescence: Slightly pubescent.
   Fragrance.—None.
Reproductive organs:
   Stamens.—Number: 4 Filament length: Approximately 1 cm.
   Anthers.—Shape: Cylindrical Length: Approximately 0.1 cm. Color: RHS Yellow-Orange 21 B Pollen: Color: RHS Yellow-Orange 21 A Quantity: Abundant.
   Pistil.—Number: 1 Length: about 1.4 cm. Style: Length: about 1.0 cm. Color: RHS Green-Yellow 1 C Stigma: Shape: Undulate Color: RHS Green-Yellow 1 B Ovary Color: RHS Green 137 C.
Other characteristics:
   Seeds and fruits.—Seeds and fruits: About 20 elliptic, small (1 mm long) beige colored seeds per one elliptic Brown capsule.
   Disease/pest resistance.—Neither resistance nor susceptibility to pathogens and pests common to Sutera have been observed.
   Temperature tolerance.—The new variety tolerates temperatures between 5 to 40° C.

What is claimed is:

1. A new and distinct cultivar of Sutera plant named ‘DANCOP37’ as herein illustrated and described.
